(19) United States
(12) Patent Application Publication
(10) Pub. No.: US 2015/0025267 A1
(43) Pub. Date: Jan. 22, 2015

(54) METAL POWDERDOUS CATALYST COMPRISING A FE-ALLOY

(71) Applicant: DSM ASSETS B.V., Heerlen (NL)

(72) Inventors: Werner Bonrath, Basel (CH); Axel Buss, Basel (CH)

(21) Appl. No.: 14/380,901

(22) PCT Filed: Feb. 22, 2013
(86) PCT No.: PCT/EP2013/053513

§ 371 Date: Aug. 25, 2014

(30) Foreign Application Priority Data
Feb. 24, 2012 (EP) 12156836.4

(51) Int. Cl. B01J 23/89; C07C 67/283; C07C 29/17

(52) U.S. Cl. CPC B01J 23/8993; C07C 29/17; C07C 67/283
USPC 560/261; 502/315; 502/185; 502/307; 568/902

(57) ABSTRACT

The present invention is related to a new metal powder catalytic system (catalyst) comprising a Fe-alloy as a carrier, its production and its use in hydrogenation processes.

The present invention is related to a new metal powder catalytic system (catalyst) comprising a Fe-alloy as a carrier, its production and its use in hydrogenation processes.

Powderous catalysts are well known and used in chemical reactions. Important types of such catalysts are i.e. the Lindlar catalysts.

A Lindlar catalyst is a heterogeneous catalyst which consists of palladium deposited on a calcium carbonate carrier which is also treated with various forms of lead.

Such catalysts are of such an importance that there is always a need for their improvement.

The goal of the present invention was to find a powderous catalyst with improved properties.

The powderous catalysts according to the present invention do have a metal (or metal alloy) as carrier material, instead of a calcium carbonate carrier.

This metal alloy is coated by a metal oxide layer on which palladium (Pd) is deposited.

Furthermore the new catalyst according to the present invention is free from lead (Pb).

Therefore, the present invention relates to a powderous catalytic system (I) comprising

a metal alloy carrier comprising
(i) 60 weight-% (wt-%)-80 wt-%, based on the total weight of the metal alloy, of Fe, and
(ii) 1 wt-%-30 wt-%, based on the total weight of the metal alloy, of Cr, and
(iii) 0.5 wt-%-10 wt-%, based on the total weight of the metal alloy, of Ni, and
   wherein the said metal alloy is coated by a metal oxide layer and impregnated with Pd.

It is obvious that all percentages always add up to 100.

This new catalyst has numerous advantages:
The catalyst is easy to recycle (and to remove) after the reaction. This can be done i.e. by filtration.
The catalyst can be used more than once (re-usable).
The catalyst as such is a very stable system. It is i.e. stable in regard to acids as well as to water.
The catalyst is easy to produce.
The catalyst is easy to handle.
The hydrogenation can be carried out without any solvents.
The catalyst is free from lead.
The catalyst shows high selectivity in hydrogenation reactions.

The catalytic system is in the form of a powder.

The metal alloy used as a carrier is preferably stainless steel.

Three main types of stainless steels are known. These are classified by their crystalline structure:

austenitic, ferritic, and martensitic.

The most important type (more than 70% of the stainless steel production volume) is the austenitic steel. Austenitic steels have austenite as their primary phase. These are alloys containing usually between 18 wt-%-20 wt-%, based on the total weight of the alloy, of chromium and 8 wt-%-10 wt-%, based on the total weight of the alloy, of nickel.

Ferritic steels have ferrite as their main phase. These steels contain iron and about 17 wt-%, based on the total weight of the alloy, of chromium.

Martensitic steel has a characteristic orthorhombic martensite microstructure. Martensitic steels are low carbon steels.

Therefore the present invention relates to a powderous catalytic system (II) wherein the metal alloy is stainless steel comprising
(I) 60 wt-%-80 wt-%, based on the total weight of the stainless steel carrier, of Fe, and
(ii) 10 wt-%-30 wt-%, based on the total weight of the stainless steel carrier, of Cr, and
(iii) 0.5 wt-%-10 wt-%, based on the total weight of the stainless steel carrier, of Ni, and
   wherein the stainless steel is coated by a metal oxide layer impregnated with Pd.

Stainless steel can comprise further metals, such as i.e. Cu, Mn, Si, Mo, Ti, Al and Nb.

Furthermore stainless steel can comprise carbon as well.

Therefore the present invention relates to a powderous catalytic system (III) wherein the metal alloy is stainless steel comprising
(i) 60 wt-%-80 wt-%, based on the total weight of the stainless steel carrier, of Fe, and
(ii) 12 wt-%-25 wt-%, based on the total weight of the stainless steel carrier, of Cr, and
(iii) 1 wt-%-8 wt-%, based on the total weight of the stainless steel, of Ni, and
(iv) 1 wt-%-8 wt-%, based on the total weight of the stainless steel carrier, of Cu, and
   wherein the stainless steel is coated by a metal oxide layer impregnated with Pd.

Stainless steel is commercially available from many producers and traders. It can be bought i.e. from companies such as Sverdrup Hanssen, Nichelcrom Acciai (fox S.p.A or EOS GmbH.

Suitable products are i.e. EOS StainlessSteel GP1® from EOS GmbH (Germany)

The metal oxide layer, which coats the metal alloy, is non-acidic (preferably basic or amphoteric). Suitable non-acidic metal oxide layers comprise Zn, Cr, Mn, Cu and/or Al. Preferably the oxide layer comprise ZnO and optionally at least one further metal oxide wherein the metal is chosen from the group consisting of Cr, Mn, Mg, Cu and Al.

Therefore the present invention also relates to a powderous catalytic system (IV), wherein powderous catalytic system (I), (II) and/or (III), the metal oxide layer is non-acidic (preferably basic or amphoteric).

Preferred is a powderous catalytic system (IV′), which is powderous catalytic system (IV), wherein the non-acidic metal oxide layer comprises Zn, Cr, Mn, Cu or Al (more preferably the oxide layer comprise ZnO and optionally at least one further metal oxide wherein the metal is chosen from the group consisting of Cr, Mn, Mg, Cu and Al).

The metal alloy is preferably coated with a thin layer of ZnO (0.5-3.5 μm thickness) and optionally at least one further metal (Cr, Mn, Mg, Cu and Al) oxide.

Therefore the present invention also relates to a powderous catalytic system (V), which is powderous catalytic system (I), (II), (III), (IV) and/or (IV′), wherein the metal alloy is coated with a thin layer of ZnO and optionally at least one further metal (Cr, Mn, Mg, Cu and/or Al) oxide.

Preferred is also a powderous catalytic system (V′), which is powderous catalytic system (V) wherein the non-acidic metal oxide layer is essentially free from Pb.

The coating of the metal alloy is done by commonly known processes, such as i.e. dip-coating.

Usually the catalytic system (catalyst) of the present invention comprises between 0.1 wt-% and 50 wt-%, based on the total weight of the catalyst, of ZnO, preferably between 0.1 wt-% and 30 wt-%, more preferably between 1.5 wt-% and 10 wt-% and most preferably between 2 wt-% and 8 wt-%.

Therefore the present invention also relates to a powderous catalytic system (VI), which is powderous catalytic system (I), (II), (III), (IV), (IV′), (V) and/or (V′), wherein the catalyst comprises between 0.1 wt-% and 50 wt-%, based on the total weight of the catalytic system, of ZnO (preferably between 0.1 wt-% and 30 wt-%, more preferably between 1.5 wt-% and 10 wt-% and most preferably between 2 wt-% and 8 wt-%).

In a preferred embodiment of the present invention the non-acidic metal oxide layers comprises ZnO and at least one further metal oxide wherein the metal is chosen from the group consisting of Cr, Mn, Mg, Cu and Al.

In a more preferred embodiment of the present the non-acidic metal oxide layer comprises ZnO and Al₂O₃.

Therefore the present invention also relates to a powderous catalytic system (VII), which is powderous catalytic system (I), (II), (III), (IV), (IV′), (V), (V′) and/or (VI), wherein the non-acidic metal oxide layer comprises ZnO and Al₂O₃.

When a mixture of ZnO and Al₂O₃ is used then it is preferred that the ratio of ZnO:Al₂O₃ is from 2:1 to 1:2 (preferably 1:1).

Therefore the present invention also relates to a powderous catalytic system (VII′), which is powderous catalytic system (VII), wherein the ratio of ZnO:Al₂O₃ is from 2:1 to 1:2 (preferably 1:1).

The coated metal alloys are then impregnated by Pd-nanoparticles. The nanoparticles are synthesized by commonly known methods, i.e. by using PdCl₂ as a precursor, which is then reduced by hydrogen.

Usually the Pd-nanoparticles, which are on the non-acidic metal oxide layer, have an average particle size of between 0.5 and 20 nm, preferably of between 2 and 15 nm, more preferably of between 5 and 12 nm and most preferably of between 7 to 10 nm. (The size is measured by light scattering methods).

Therefore the present invention also relates to a powderous catalytic system (VIII), which is powderous catalytic system (I), (II), (III), (IV), (IV′), (V), (V′). (VI), (VII) and/or (VII′), wherein the Pd-nanoparticles have an average particle size of between 0.5 and 20 nm (preferably of between 2 and 15 nm, more preferably of between 5 and 12 nm and most preferably of between 7 to 10 nm).

The catalyst according to present invention comprises between 0.001 wt-% and 5 wt-%, based on the total weight of the catalyst, of the Pd— nanoparticles, preferably between 0.01 wt-% and 2 wt-% more preferably between 0.05 wt-% and 1 wt-%.

Therefore the present invention also relates to a powderous catalytic system (IX), which is powderous catalytic system (I), (II), (III), (IV), (IV′), (V), (V′), (VI), (VII), (VII′) and/or (VIII), wherein the catalyst comprises between 0.001 wt-% and 5 wt-%, based on the total weight of the catalyst, of the Pd— nanoparticles (preferably between 0.01 wt-% and 2 wt-% more preferably between 0.05 wt-% and 1 wt-%).

The catalyst is usually activated before the use. The activation is done by using well known processes, such thermo activation in H₂.

The catalyst of the present invention is used in selective catalytic hydrogenation of organic starting material, especially of organic starting material comprising a carbon-carbon triple bond, more especially of alkynol compounds.

Therefore the present invention also relates to the use of a powderous catalytic system (catalyst) (I), (II), (III), (IV), (IV′), (V), (V′), (VI), (VII), (VII′), (VIII) and/or (IX) in selective catalytic hydrogenation of organic starting material, especially of organic starting material comprising a carbon-carbon triple bond, more especially of alkynol compounds.

Preferably the present invention relates to a process of reacting a compound of formula (I)

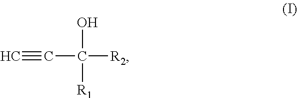

wherein
R₁ is linear or branched C₅-C₃₅ alkyl or linear or branched C₅-C₃₅ alkenyl moiety, wherein the C chain can be substituted, and
R₂ is linear or branched C₁-C₄ alkyl, wherein the C chain can be substituted,
   with hydrogen in the presence of a catalyst (I), (II), (III), (IV) (IV′), (V), (V′), (VI), (VII) (VII′), (VIII) and/or (IX).

Hydrogen is usually used in the form H₂ gas.

Preferred compounds of formula (I) are the following:

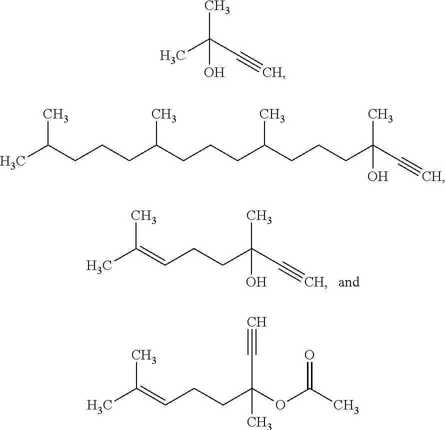

The following examples serve to illustrate the invention. All percentages are related to weight and the temperatures are given in degree Celsius, if not otherwise stated.

### EXAMPLES

### Example 1

### Synthesis of the Catalyst (Stainless Steel Coated by al₂O₃/ZnO and Pd Deposition)

### Step 1: Thermal Pre-Treatment

The stainless steel powder (EOS StainlessSteel GP1® commercially available from EOS GmbH, Germany) was subjected to a thermal pre-treatment at 450° C. for 3 h.

### Step 2 Deposition of ZnO+Al₂O₃ (Coating of the Metal Alloy Carrier)

To a 100 ml-flask 20.0 g (53.3 mMol) of Al(NO₃)₃ 9H₂O and 70 ml of water were added. The mixture was stirred until the Al(NO₃)₃.9H₂O was completely dissolved. The solution was heated up to 95° C. Then 4.34 g (53.3 mMol) of ZnO powder was slowly added to the reaction solution. Heating and stirring were maintained until the ZnO was completely dissolved. The solution was then cooled down to room temperature and filtrated through a membrane filter.

The deposition of ZnO/Al₂O₃ was performed by adding the oxidized stainless steel powder (23.4 g) from step 1 to the precursor solution and stirring the mixture at room temperature for 15 min.

The powder was then filtered off via a membrane filter and dried in air at 40° C. and 125 mbar for 2 h followed by a calcination step at 450° C. for 1 h. The stirring-drying-calcination cycle was repeated 3 times. Finally, the powder support was calcined in air at 550° C. for 1 h.

22.75 g of coated stainless steel powder was obtained.

### Step 3: Preparation and Deposition of the Pd-Nanoparticles

318 mg (1.31 mmol) of sodium molybdate dihydrate and 212 mg (1.20 mmol) of palladium(11) chloride anhydrous were added to 60 ml of deionized water under heating (ca. 95° C.). The mixture was stirred. The heating and stirring were continued until complete evaporation of the water (solid residue was formed). Afterwards, 60 ml of deionized water were added to the residue under stirring. The evaporation-dissolving cycle was repeated two times in order to completely dissolve PdCl₂. Finally, 100 ml of hot water were added to the solid residue. The deep brown solution was cooled down to room temperature and filtrated through a paper filter. The filter was washed with water until the final volume of the precursor solution was 120 mL.

Afterwards the Pd° suspension was formed by bubbling hydrogen through the precursor solution for 1 h in a glass cylinder at room temperature.

The so obtained Pd° suspension and 22.75 g of the coated stainless steel powder (from step 2) were added to a 200 ml-flask. The mixture was stirred at room temperature for 15 min. The powder was filtered off via a filter paper and dried in air at 40° C. and 125 mbar for 2 h. This process was repeated twice.

### Step 4: Thermo Activation of the Catalyst in H₂

The powder catalyst obtained from step 3 was subjected to a temperature treatment at 300° C. for 4 h under H₂—Ar flow. Then, it was cooled down to room temperature under the same H₂—Ar flow.

20.3 g of the powderous catalyst according to the present invention was obtained.

### Example 2a

### Selective Hydrogenation of MBY to MBE

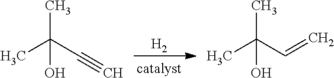

To 285 g (3.38 Mol) of MBY 1.5 g of the catalyst of Example 1 was added under stirring. The reaction was carried out at 45° C. and 4 bar pressure.

The reaction was repeated four times under the same condition.

At the end of the reaction (after about 23 hours) the selectivity of the reaction was between 91.6 and 95.6% and the conversion was between 99.4 and 99.9%.

It can be seen that the new powderous catalyst has excellent properties as a catalyst for selective hydrogenations.

### Example 2b

### Repeated Selective Hydrogenation of MBY to MBE

The same reaction conditions as in Example 2a have been used. At the end of the reaction (after about 13-19 hours), the reaction mixture was cooled down under inter atmosphere and the reaction solution was exchanged with new MBY (again 285 g) and the hydrogenation was started again.

7 cycles have been run. The following table shows the results of the cycles.

| | Selectivity | Conversion | Yield |
|---|---|---|---|
| Cycles | [%] | [%] | [%] |
| 1 | 96.17 | 99.78 | 96.0 |
| 2 | 96.03 | 99.99 | 96.0 |
| 3 | 95.72 | 99.98 | 95.7 |
| 4 | 95.65 | 99.99 | 95.6 |
| 5 | 96.01 | 99.99 | 96.0 |
| 6 | 96.07 | 99.98 | 96.1 |
| 7 | 95.99 | 99.99 | 96.0 |

It can be seen that the new powderous catalyst keeps the excellent catalytic properties even after 7 cycles (without treating the catalyst after each cycle).

### Example 3

### Selective Hydrogenation of Dehydrolinalool (DLL)

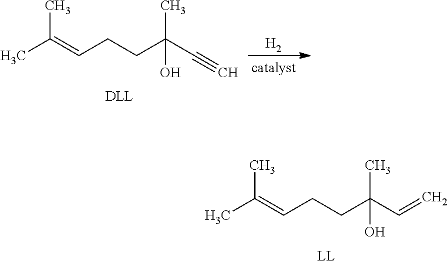

To 285 g (1.87 Mol) of DLL 1.5 g of the catalyst of Example 1 was added under stirring. The reaction was carried out at 55° C. and 4 bar pressure for about 9 hours.

At the end of the reaction the selectivity of the reaction was 94.1% and the conversion was 98.09%.

It can be seen that the new powderous catalyst has excellent properties as a catalyst for selective hydrogenations.

### Example 4

### Selective Hydrogenation of Dehydrolinalyl Acetate (DLA)

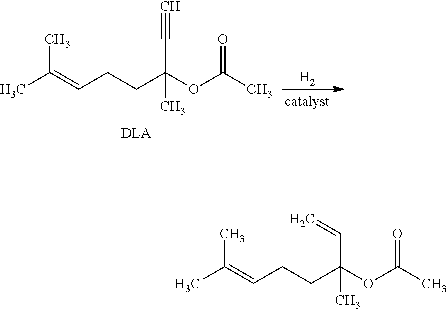

To 285 g (1.5 Mol) of DLA 1.5 g of the catalyst of Example 1 was added under stirring. The reaction was carried out at 40° C. and 4 bar pressure for about 34 hours.

At the end of the reaction the selectivity of the reaction was 89.53% and the conversion was 98.67%.

### Example 5

### Selective Hydrogenation of Dehydroisophytol (DIP)

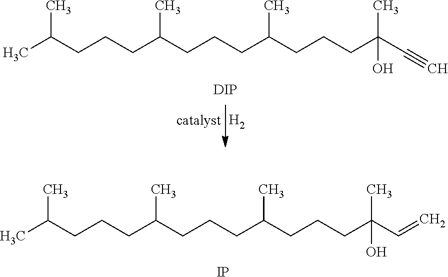

To 285 g (0.97 Mol) of DIP 1.5 g of the catalyst of Example 1 was added under stirring. The reaction was carried out at 85° C. and 4 bar pressure for about 5.5 hours.

At the end of the reaction the selectivity of the reaction was 87.90% and the conversion was 94.33%.

It can be seen that the new powderous catalyst has excellent properties as a catalyst for selective hydrogenations.

1. A powderous catalytic system comprising
a metal alloy carrier comprising
(i) 60 wt-%-80 wt-%, based on the total weight of the metal alloy, of Fe, and
(ii) 1 wt-%-30 wt-%, based on the total weight of the metal alloy, of Cr, and
(iii) 0.5 wt-%-10 wt-%, based on the total weight of the metal alloy, of Ni, and
wherein the said metal alloy
is coated by a metal oxide layer and impregnated with Pd.

2. Catalyst according to claim 1, wherein the metal alloy is stainless steel.

3. Catalyst according to claim wherein the metal alloy comprises further metals.

4. Catalyst according to claim 1, wherein the metal alloy comprises carbon.

5. Catalyst according to claim 1, wherein the metal alloy is stainless steel comprising
(i) 60 wt-%-80 wt-%, based on the total weight of the stainless steel, of Fe, and
(ii) 12 wt-%-25 wt-%, based on the total weight of the stainless steel, of Cr, and
(iii) 1 wt-%-8 wt-%, based on the total weight of the stainless steel, of Ni, and
(iv) 1 wt-%-8 wt-%, based on the total weight of the stainless steel, of Cu, and
wherein the said metal alloy is coated by a metal oxide layer and impregnated with Pd.

6. Catalyst according to claim 1, wherein the metal oxide layer is basic or amphoteric.

7. Catalyst according to claim 1, wherein the metal oxide layer comprises Zn, Cr, Mn, Cu and/or Al.

8. Catalyst according to claim 1, wherein the oxide layer comprises ZnO and optionally at least one further metal oxide chosen from the group consisting of Cr, Mn, Mg, Cu and Al.

9. Catalyst according to claim 1, wherein the oxide layer comprises ZnO and Al₂O₃.

10. Catalyst according to claim 1 comprising between 0.1 wt-% and 50 wt-%, based on the total weight of the catalyst, of the non acidic metal oxide layer.

11. Catalyst according to claim 1, wherein the metal oxide is mixture of ZnO and Al₂O₃ in a ratio of 2:1 to 1:2 (preferably 1:1).

12. Catalyst according to claim 1, wherein the Pd-nanoparticles have an average particle size of between 0.5 and 20 nm.

13. Catalyst according to claim 1, wherein the catalyst comprises between 0.001 wt-% and 5 wt-%, based on the total weight of the catalyst, of the Pd— nanoparticles.

14. Use of a catalyst according to claim 1 in selective catalytic hydrogenation of organic starting material.

15. Use according to claim 14, wherein the organic starting material is a compound of formula (I) 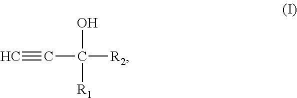
wherein
R₁ is linear or branched C₅-C₃₅ alkyl or linear or branched C₅-C₃₅ alkenyl moiety, wherein the C chain can be substituted, and
R₂ is linear or branched C₁-C₄ alkyl, wherein the C chain can be substituted.
